(19) United States
(12) Patent Application Publication
(10) Pub. No.: US 2003/0017583 A1
(43) Pub. Date: Jan. 23, 2003

(54) Method of disposal of pet waste

(76) Inventor: Carl Mitchell, Castleton (CA)

Correspondence Address: BIRCH STEWART KOLASCH & BIRCH, PO BOX 747, FALLS CHURCH, VA, 22040-0747, US

(21) Appl. No.: 10/125,429

(22) Filed: Apr. 19, 2002

(30) Foreign Application Priority Data
Apr. 19, 2001 (CA) 2,344,512

(51) Int. Cl. C12M 1/00

(52) U.S. Cl. 435/290.4

(57) ABSTRACT

A method of collecting excrement of pets such as dogs and cats and disposing of it in an environmentally acceptable manner, The method involves the steps of: providing a number of receptacles for excrement in a household containing pets, a park and wherever else there are pets accompanied by humans. Receptacles containing excrement are periodically collected and transported to a facility for disposal. At the facility the excrement is disposed of by flushing it into the sewer where that is legal or by composting by means of worms, larvae, micro-organisms, heat or the combination of heat, wind and rain.

### BACKGROUND OF THE INVENTION

This invention relates to the collection and disposal of organic waste and more particularly to the collection of excrement of pets and of disposal of the excrement in a number of environmentally acceptable ways.

The disposal of excrement of pets such as dogs, cats, rabbits and birds is becoming more and more a problem as the concern for the environment grows in society. At one time it was acceptable to leave dogs' faeces on the ground to decompose naturally but that is no longer generally acceptable. As for cats, their faeces could be added to other household garbage and left out for pickup by municipal garbage collectors. However many municipalities are monitoring the volume of garbage left for pickup and are charging according to that volume. A cat can generate a significant amount of waste, particularly where it is mixed with clumping material in a litter box. The cost of pickup of such waste can be significant.

While the disposal of excrement of rabbits, birds and small rodents is not generally a problem, the excrement does attract flies and has an objectionable smell.

### SUMMARY OF THE INVENTION

I have devised a method of collecting excrement of pets from households and treating it so that it is acceptable for disposal in the ground, at land fill sites and so on. The method includes the steps of collection of such excrement from receptacles in residences, parks and wherever else there are receptacles for such excrement; and composting the excrement in order to reduce its pathogen content to a level legally acceptable for disposal. My method obviates many of the disadvantages of the conventional methods of collection and disposal of excrement. For example, according to my method, before the excrement is picked up, it is stored in a bag that is largely free of odour and is protected from flies. After pickup, the excrement is disposed of in a way that is environmentally acceptable.

Excrement treated by my method can be diverted from a municipal waste disposal facility and can be treated in a way that is environmentally acceptable and in a way that relieves the pet owner of all responsibility for it after it has been collected.

Briefly, my method involves the steps of: (i) providing a plurality of receptacles for the excrement of pets; (ii) periodically collecting receptacles containing such excrement and transporting the excrement-containing receptacles to a facility for disposal thereof; (iii) discharging the contents of the excrement-containing receptacles at the facility; and (iv) disposal of the contents in an environmentally acceptable manner.

Alternatively, my method involves the steps of: (i) providing a plurality of receptacles for the excrement of pets; (ii) periodically collecting excrement-containing receptacles and transporting the excrement-containing receptacles to a facility for composting thereof; (iii) opening the excrement-containing receptacles at the facility and discharging the contents to permit composting thereof; and (iv) composting the contents by means of worms, larvae, micro-organisms or heat to reduce its pathogen content to a level acceptable for disposal into the environment.

### DESCRIPTION OF THE DRAWINGS

The method of the invention is described with reference to the accompanying drawings in which:

FIG. 1 is a flow chart which shows the sequence of steps of the method of my invention; and

FIG. 2 is a perspective view, partly cut away, of a receptacle for use in my method.

### DESCRIPTION OF THE PREFERRED METHOD

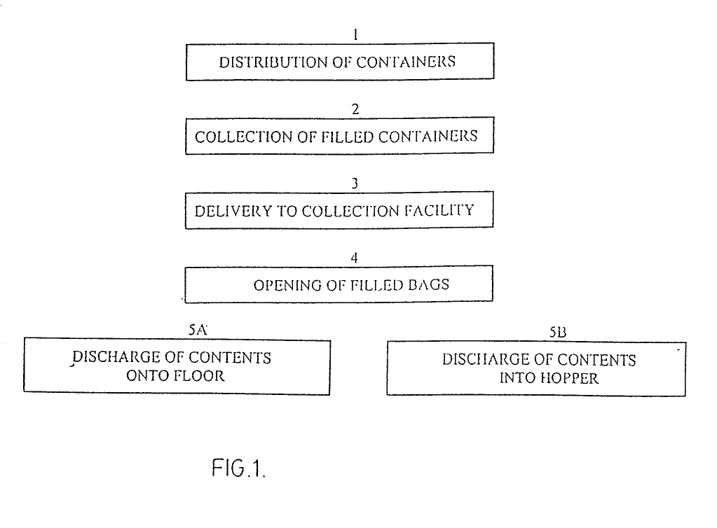

With reference to FIG. 1, the first step of the method of the invention, indicated 1, is to distribute receptacles to households with pets such as dogs, cats, rabbits, gerbils and birds. The receptacles may also be distributed to parks and wherever else there are pets accompanied by humans. The receptacles contain bags composed of plastic or of biodegradable material that will retain its integrity until the contents of the bag are ready for disposal. Kraft paper is a suitable biodegradable material. The receptacles are intended to hold the excrement-containing bags until they are collected. The receptacles may be colour-coded in order to distinguish them from receptacles containing recyclables such as cans and bottles.

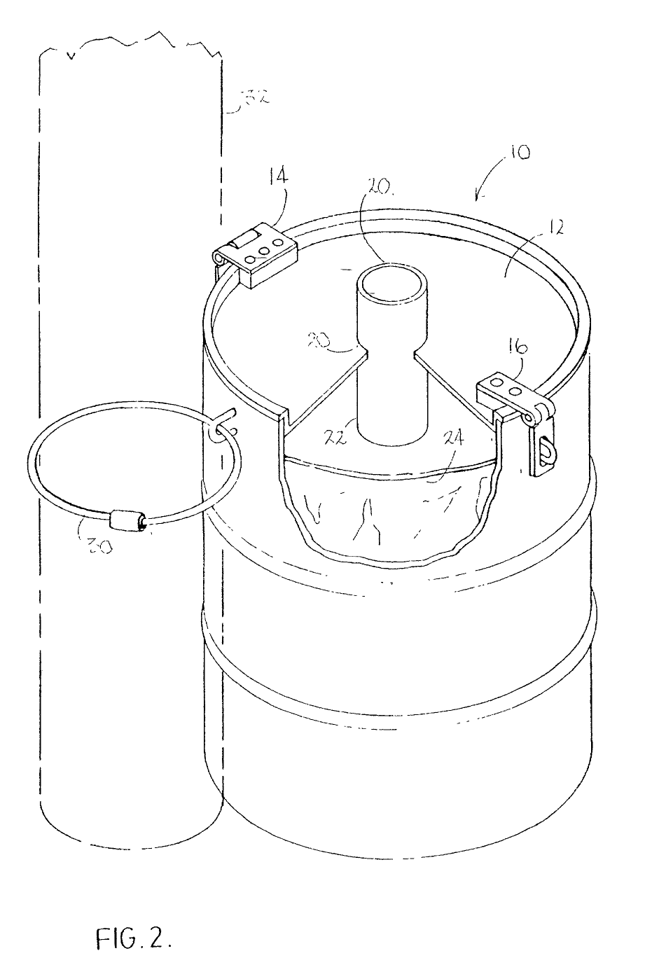

With reference to FIG. 2, the receptacle is composed of a barrel 10 such as a 45 gallon oil drum. The barrel has a lid 12 to which is attached a hinge 14 at one side and a hasp 16 at the diametrically opposite side. A padlock (not illustrated) connects the hasp to the barrel so that the lid can only be opened by unlocking the padlock. The padlock discourages vandals from overturning the barrel and strewing its contents onto the ground.

An aperture 20 is formed centrally of the lid and the aperture receives a pipe 22 which extends downwardly into the barrel. A bag 24 is removably contained within the barrel and is secured open by means of spring clips or other means to the interior wall of the barrel so that material dropped through aperture 20 falls through the pipe and into the bag.

The pipe is preferably about 20 cm. in diameter and about 20 cm. long and as such will help to protect the receptacle from vandalism because the pipe will starve lighted matches thrown into the receptacle of oxygen.

A ring 30 is attached to the receptacle and to a hydro pole 32 to discourage theft of the barrel.

Excrement from pets, in most cases in small bags collected by the pet owners, is dropped into larger bags within the receptacles for later collection. With reference again to FIG. 2, the next step 2 is to collect the larger bags within the receptacles. Collection is periodic in the same way as garbage is conventionally collected by municipalities. The bags are transported to a collection facility for disposal at step 3.

At step 4, the bags, if composed of non-biodegradable material such as plastic, are opened at the disposal facility, either by hand or mechanically, and the contents of the bags are discharged either onto the floor of the collection facility at step 5A or into a hopper at step 5B. If the bags are biodegradable they need not be opened before their contents are discharged.

Disposal of the excrement can be carried out in various ways. If the excrement is on the floor of the collection facility, it can be picked up by a front-end loader and transported to a field where it is formed into long piles or windrows. The excrement will be exposed to the sun, wind and rain in the windrows and break down into a product which is wholly or largely free of pathogens. The resulting product can be incinerated, used as a fertilizer, turned into the soil and so on.

Excrement picked up by the loader can alternatively be conveyed to a furnace or a microwave oven. Heating of the excrement is an effective way to cause the excrement to degrade while simultaneously destroying pathogens. Furnaces and ovens suitable for this purpose are well known and no description of them is deemed necessary.

Another method of disposal of the excrement is simply to flush it into a sewer or transporting it to a landfill site. Local regulations must of course permit such a method of disposal.

Excrement that is discharged into a hopper at step 5B can be composted by vermior larval-composting or by means of micro-organisms. It is usually desirable, prior to composting, to add enzymes for accelerating the breakdown of the cellular structure of the excrement to enable it to be digested by worms, larvae, or micro-organisms. U.S. Pat. No. 4,032,318 to Lovness describes a method by which this can be accomplished. The patent also describes a method for facilitating decomposition by means of micro-organisms.

Vermicomposting is effective in reducing such pathogens as fecal coliform, Salmonella sp., enteric virus and helminth ova. A process suitable for composting by means of worms involves discharging the excrement upon a substrate of earth of about 15 cm in depth and seeded with E. foetida at 1:1.5 earthworm biomass to earth ratio. Over time, the worms will migrate to the excrement and ingest it. The worm's excrement will have a substantially lower level of pathogens, in fact the excrement may be suitable as fertilizer or as soil. When the pathogen level of the thus-treated excrement has reached an acceptable value, the material may be dried by windrowing or in a conventional drier.

Worms suitable for the process include those of the families Lumbricideae, Eiseniae, Allolobophorea, Dendrobaenae and African Night-crawler.

Another method of vermicomposting involves discharging the excrement into a blend of composted manure, peat moss and soil. Such as blend is commercially available under the trade-mark “Triple-Mix” and is said to contain manure, peat moss and soil in approximately equal volumes. The weight of excrement added to the blend should preferably not exceed one-third of the weight of the blend.

The excrement and blend should then be mixed and sufficient water added until the mixture is damp. It should not be so wet that it becomes a slurry since worms may not be able to survive in a slurry. Worms such as red wrigglers (Eisenia Foetida) are then added to the mixture to compost the excrement. I have found that if the mixture is added to a receptacle, 60 cm. long by 30 cm. wide by 60 cm. in height, 10 worms will turn the mixture into a fully or substantially fully composted product in about three months. The receptacle should be vented to allow air to circulate around the mixture and should preferably be closed since worms prefer the dark Unpleasant odours will also be reduced in a closed receptacle. Ambient temperature can vary from about 4 degrees C. to about 30 degrees C. during that time as long as the mixture remains damp.

Other vermicomposting methods such are described in U.S. Pat. No. 5,082,486 to Glogowski may also be useful in reducing the pathogen level in the excrement.

Larval-composting is quite effective in eliminating pathogens from excrement. It however is quite slow and gives off a foul odour while it is taking place. The process simply involves placing the excrement in a closed receptacle and introducing into the receptacle those insects which consume excrement. The insects and their larvae will, over time, convert the excrement into a product having a level of pathogens substantially less than that of the original excrement.

Certain larvae such as maggots prefer to work in a damp environment. The air within the receptacle should accordingly be humidified or water should be added to the excrement before or during the time that the larvae are in the receptacle. Oxygenating gas may also be introduced into the receptacle to speed up the composting.

The receptacle will of course remain closed during composting to prevent the insects and larvae from escaping.

Composting by the methods described above is continued until the level of pathogens is within an acceptable range. For example, the EPA's Class A pathogen requirement is as follows: The density of fecal coliform in the compost must be less than 1,000 most probable numbers (MPN/gram total solids (dry-weight basis)) or the density of Salmonella sp. bacteria in the compost must be less than 3 MPN/4 grams of total solids (dry weight-basis).

Once the level of pathogens in the compost has been reduced to the required value, the compost can be used as a fertilizer, as soil or it can be simply turned into the existing soil.

Another method of composting the excrement is to subject it to anaerobic digestion. The product resulting from such a process can be in the form of a methane fertilizer and a liquid effluent. U.S. Pat. Nos. 4,503,154 and No. 4,429,043 both to Paton describe methods for accomplishing this.

It will be understood of course that modification can be made in my method without departing from the scope and purview of my invention as described and claimed herein.

I claim:

1. A method of collecting excrement of pets and of disposing of said excrement in a manner environmentally acceptable including the steps of: (i) providing a plurality of receptacles for said excrement; (ii) periodically collecting said receptacles containing excrement and transporting said excrement-containing receptacles to a facility for disposal thereof; (iii) discharging said excrement-containing receptacles at said facility; and (iv) disposal of said contents in an environmentally acceptable manner.

2. A method of collecting excrement of pets and of composting said excrement to reduce the pathogen content thereof to a level legally acceptable for disposal into the environment including the steps of: (i) providing a plurality of receptacles for said excrement; (ii) periodically collecting said receptacles containing excrement and transporting said excrement-containing receptacles to a facility for disposal thereof; (iii) discharging said excrement-containing receptacles at said facility to permit composting thereof; and (iv) composting said contents to reduce the pathogen content thereof to a level legally acceptable for disposal into the environment.

3. The method of claim 2 wherein composting according to step (iv) is carried out by means of worms.

4. The method of claim 2 wherein composting according to step (iv) is carried out by means of larvae.

5. The method of claim 2 wherein composting according to step (iv) is carried out by means of micro-organisms.

6. The method of claim 2 wherein composting according to step (iv) is carried out by means of heat.

7. The method of claim 3 wherein composting according to step (iv) includes the step of discharging said contents of step (iii) upon a substrate of earth of about 15 cm in depth and seeded with E. foetida at 1:1.5 earthworm biomass to earth ratio; and allowing sufficient time for the earthworms to migrate to and digest said excrement.

8. The method of claim 3 wherein composting according to step (iv) includes the step of discharging said contents of step (iii) into a blend which contains manure, peat moss and soil; combining said contents and blend to form a mixture; adjusting the moisture content until said mixture is damp; adding the earthworms Eisenia Foetida to said damp mixture; and allowing sufficient time for the earthworms to migrate to and digest said excrement.

9. The method of claim 8 further including adjusting the content of said blend such that the ratio of manure, peat moss and soil is approximately 1:1:1 by volume.

10. The method of claim 9 wherein the ratio of excrement and blend in said mixture is approximately 1:3 by weight.

11. The method of claim 10 wherein the temperature of said damp mixture at the time said earthworms are allowed to migrate to said damp mixture and digest said excrement is maintained in the range of about 4 degrees C. to about 30 degrees C.
